(19) United States
(12) Patent Application Publication
(10) Pub. No.: US 2009/0163410 A1
(43) Pub. Date: Jun. 25, 2009

(54) Novel Peptides and the Biological Use Thereof

(76) Inventors: Daniel Baty, Marseille (FR); Yves Collete, Carnoux En Provence (FR); Francoise Guerlesquin, Marseille (FR); Xavier Morelli, Marseille (FR); Isabelle Parrot, Beaulieu (FR); Stefan Arold, Montpellier (FR); Serge Benichou, Paris (FR)

Correspondence Address: NIXON & VANDERHYE, PC, 901 NORTH GLEBE ROAD, 11TH FLOOR, ARLINGTON, VA, 22203, US

(21) Appl. No.: 12/086,243

(22) PCT Filed: Dec. 11, 2006
(86) PCT No.: PCT/FR2006/002697

§ 371 Date: Nov. 12, 2008

(30) Foreign Application Priority Data
Dec. 9, 2005 (FR) 0512524

(51) Int. Cl. A61K 38/16; C07K 14/00; A61K 38/08; A61K 38/10; C07K 7/04; A61P 31/18

(52) U.S. Cl. 514/12; 530/324; 514/17; 514/15; 514/16; 514/13; 514/14; 530/326; 530/327; 530/328; 530/329

(57) ABSTRACT

The invention relates to novel purified insulated peptides, which exhibit, in particular, Nef protein-binding properties and are characterised in that they contain an amino acid sequence of a formula SEQ ID No 1: W-P-a-W-L-P, wherein a is selected from W, A, S or D.

The invention relates to novel purified, isolated peptides which bind in particular the Nef protein of HIV-1. It also relates to the uses thereof as inhibitors of the interactions of Nef with its partners in infected cells and, in this respect, as antiretroviral medicaments.

The virulence of HIV comes from its substantial replicative capacity and also its pathogenic nature, demonstrated in animal models not permissive to viral replication. Several viral gene products contribute directly and indirectly to the pathogenicity, and are involved in the development of an acquired immunodeficiency syndrome (AIDS).

Among these proteins, Nef constitutes a target of interest. Many cell partners of Nef have been identified, including SH3-domaine cell proteins. The involvement of Nef in the viral cycle and its important role therefore make it the target of choice against which no known inhibitor exists at this time. All these arguments have led the inventors to propose the Nef protein as a viral target of major importance, and to develop inhibitors capable of interfering with its biological functions, and by extension, on the one hand, with the replication and pathogenicity of the HIV-1 virus, and on the other hand, with the immunogenicity of the infected cells.

In particular, the demonstration of a consensus sequence in peptides that have been obtained by the phage-display technique and have been characterized makes it possible to have Nef-inhibiting compounds of great value and provides the means for developing an approach for modeling drugs that target the complementary molecular surfaces between the Nef protein and the peptides.

The objective of the invention is therefore to provide novel peptides capable of specifically targeting Nef regions involved in HIV-1 infection.

The invention is also directed toward providing means for obtaining such peptides.

The objective of the invention is also to take advantage of the Nef-inhibiting properties of these peptides and it is directed toward the therapeutic applications thereof, more especially for the treatment of HIV-1 infections.

The purified isolated peptides of the invention are characterized in that they contain an amino acid sequence corresponding to SEQ ID No. 1:

W-P-a-W-L-P

wherein
a is selected from W, A, S or D.

Peptides of the invention which have this sequence correspond to the amino acid sequence SEQ ID No. 2:

b-W-P-a-W-L-P-c-d-f

wherein
b=R/T or is absent
a is as defined above
c=Q, T, L, C or H or is absent
d=L, W or A or is absent
f=P or is absent.

Peptides of this group are decameric peptides and correspond to the following sequences SEQ ID No. 3 to SEQ ID No. 7:

| | |
|---|---|
| SEQ ID No. 3: | N T W P W W L P T L |
| SEQ ID No. 4: | Y R W P A W L P L W |
| SEQ ID No. 5: | N W R W P W W I P G |
| SEQ ID No. 6: | T W P W W L P H A P |
| SEQ ID No. 7: | W P S W L P Q L P F |

Advantageously, these peptides bind to Nef with an affinity of the order of one micromolar.

Other peptides correspond to the following sequences SEQ ID No. 8 to SEQ ID No. 13:

| | |
|---|---|
| SEQ ID No. 8: | W P S W L P Q |
| SEQ ID No. 9: | W P S W L P |
| SEQ ID NO. 10: | W P W W L P |
| SEQ ID No. 11: | W P A W L P |
| SEQ ID No. 12: | W P D W L P |
| SEQ ID No. 13: | W P S W L P Q L P |

According to one embodiment of the invention, the peptides defined above may contain an amino acid sequence comprising, where appropriate, amino acid derivatives which facilitate the penetration of said peptides into cells.

Derived peptides of this type contain, for example, at one of their ends, a sequence selected from: (R)ₙ with n=6 to 8; R (A R R)ₙ₁, with n1=1 to 3; R(Ahx R)ₙ₂, with n2=1 to 6; SEQ ID No. 14: K K R R Q R R R; and SEQ ID No. 15: R Q I K I W F Q N R Nle K W K K.

In these sequences, “Ahx” represents an aminohexanoic acid unit and “Nle” represents norleucine,

The invention is in particular directed toward peptides derived from sequence ID No. 11, corresponding to the following sequences SEQ ID No. 16 to SEQ ID No. 21:

| | |
|---|---|
| SEQ ID No. 16: | R R R R R R W P A W L P |
| SEQ ID No. 17: | R R R R R R R R W P A W L P |
| SEQ ID No. 18: | R A R R A R R A R R W P A W L P |
| SEQ ID No. 19: | R Ahx R Ahx R Ahx R Ahx R Ahx R Ahx |
| | R W P A W L P |
| SEQ ID No. 20: | K K R R Q R R R W P A W L P |
| SEQ ID No. 21: | R Q I K I W F Q N R Nle K W K K W P |
| | A W L P. |

The peptides of the invention which bind the Nef protein are readily obtained by carrying out conventional peptide synthesis techniques and constitute pure products.

The peptides of the invention bind to a molecular surface of Nef that is involved in the interaction of the latter with the SH3 domain of Hck and the PAK kinase, and is required for the functions of modulation of the surface expression of MHC class 1 molecules and the increase in viral infectivity due to Nef.

These peptides are therefore tools of choice to be used as inhibitors of the interaction between Nef and some of its cellular partners, including SH3-domain proteins. They can also be used to develop chemical molecules on the basis of their amino acid sequences and/or of their structural data, and, in this application, said peptides are used directly or are fused to elements that facilitate their penetration into cells.

They in fact make it possible to develop molecules (modified peptides or chemical molecules) which inhibit the interaction of Nef with some of its cellular partners, including SH3-domain proteins, and which therefore have effects on the cell and viral functions modulated by Nef via these interactions, in particular the propagation of the HIV-1 virus in the infected host.

Given their properties, the peptides of the invention are particularly suitable for constituting active ingredients of antiviral medicaments.

The invention is therefore also directed toward pharmaceutical compositions characterized in that they comprise a therapeutically effective amount of at least one peptide as defined above, combined with pharmacologically acceptable excipients.

These compositions are in forms suitable for their administration for the purpose of an anti-HIV treatment. They are advantageously injectable compositions containing said peptides in solution or in suspension. Such compositions contain, for example, from 10 μg to 50 mg of peptide.

Other characteristics and advantages of the invention are given in the examples which follow, which comprise references to FIGS. 1 to 7, representing respectively:

FIG. 1: the alignment of the Nef_{Δ1-57}-binding peptides,

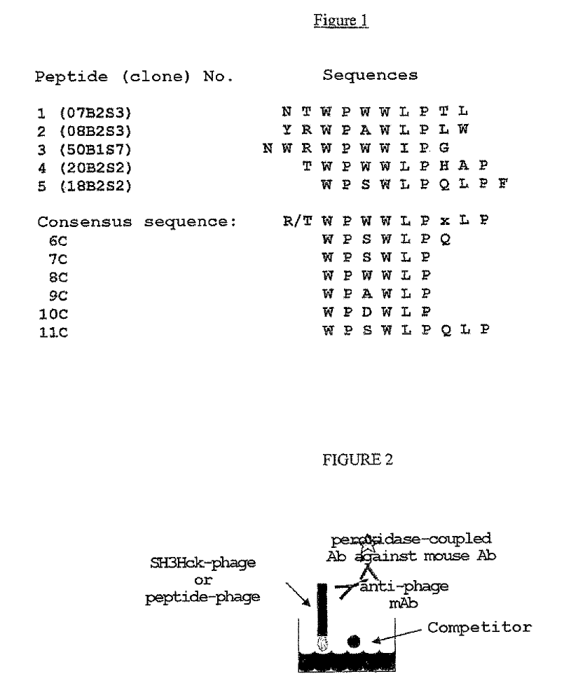

FIG. 2: the performing of ELISA assays for measuring the interaction between Nef and the various partners, the SH3Hck-phage or the peptide phages derived from a peptide library,

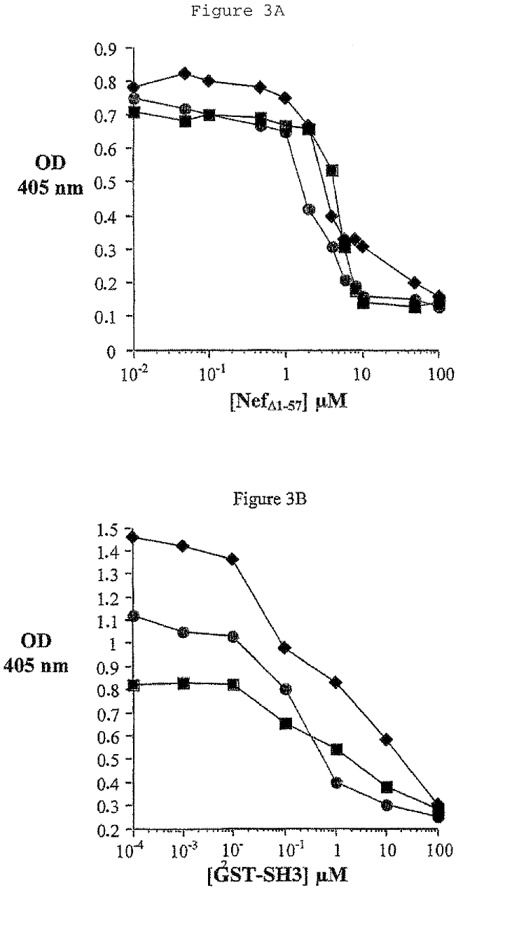

FIG. 3: displacement of the peptide-phages by Nef_{Δ1-57} and GST-SH3,

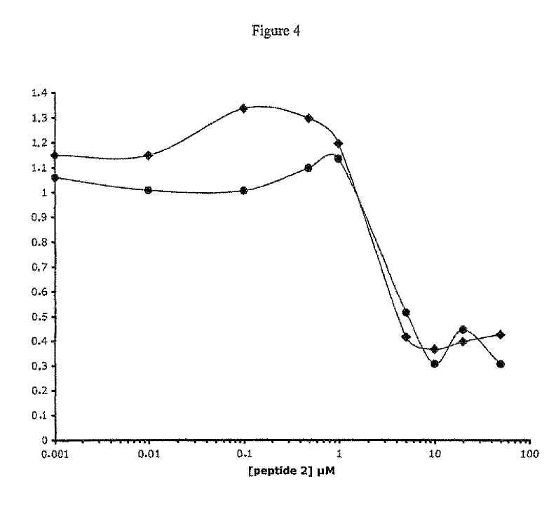

FIG. 4: displacement of the phages by synthetic peptides,

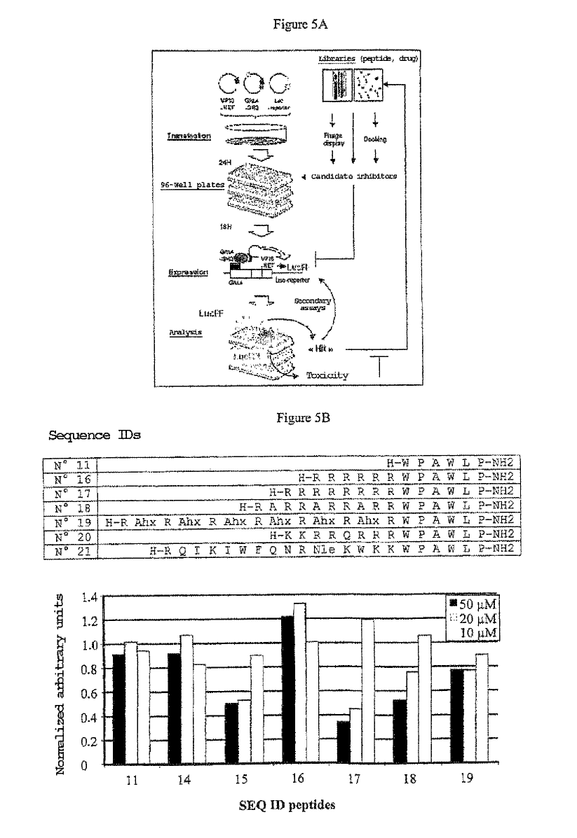

FIG. 5: the cellular activity of the peptides,

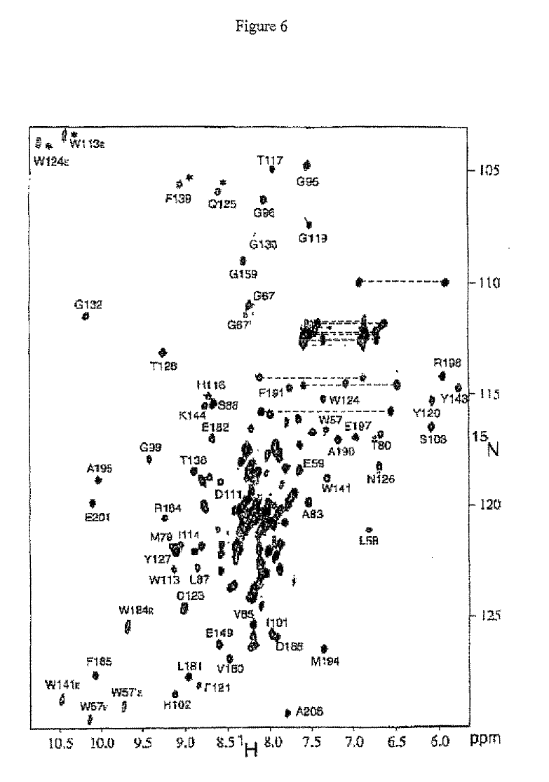

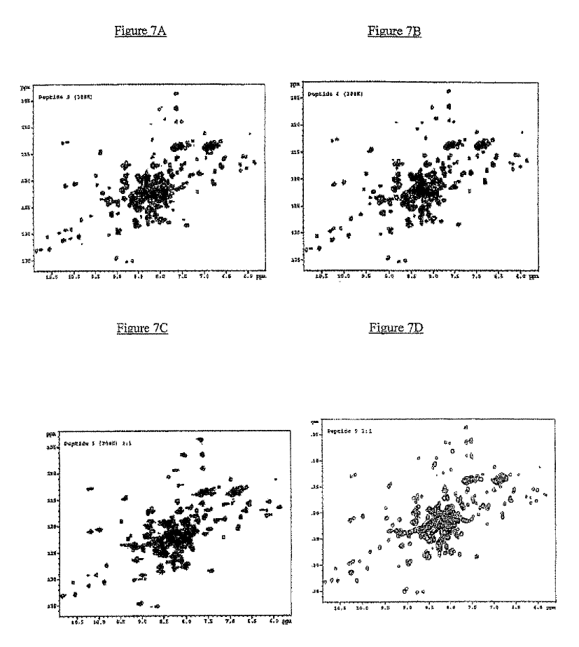

FIG. 6: the ¹H-¹⁵N HSQC spectrum, and

FIG. 7: the HSQC spectra for Nef.

### The Nef Protein

The Nef protein (Nef HIV-1 LAI) used is a recombinant protein purified from a GST-Nef_{Δ1-57} fusion protein after cleavage with thrombin (Arold et al., 1997). The Nef_{Δ1-57} protein has had residues 1 to 57 deleted because this region is not structured in solution and had to be cleaved so as to allow crystals to be obtained in order to resolve the structure of the protein.

In order to carry out the heteronuclear NMR experiments, the Nef protein is enriched in ¹⁵N by producing it in E. coli cultured on MD minimum medium in which the ammonium chloride is substituted with ¹⁵NH₄Cl (Eurisotop).

1. Construction of a control phage expressing at its surface the RT region of the SH3 domain of Hck

The DNA region encoding residues 62 to 118 of the SH3 domain of Hck was amplified by PCR from the plasmid pBindHckSH3 and was cloned into the phagemid vector phenyl (Hoogenboom et al., 1991) between the PstI and EagI restriction enzyme sites. In the presence of a helper phage, this phagemid makes it possible to express the SH3 domain fused to the N-terminal of protein-3 (p3) at the head of the M13 phage.

Sequences SEQ ID Nos 22 and 23 of the oligonucleotides used:

5′ SH3/PstI
SEQ ID No. 22:
AATGCAAAACTGCAGGTGGTTGCCCTGTATG
3′ SH3/EagI
SEQ ID No. 23:
TTTGTTCTGCGGCCGCGTCAACGCGGGCGAC

PCR1 Conditions:

The fragment encoding the SH3 domain of Hck was amplified by PCR using 0.1 μl of the plasmid pBindHckSH3 with 0.5 U of Dynazyme Extend DNA polymerase(Finnzymes), 10 pmol of the 5′ SH3/PstI primer and 10 pmol of the 3′SH3/EagI primer, in a volume of 50 μl (94° C., 3 min; 94° C., 1 min; 60° C., 1 min; 72° C., 1 min; 37 cycles, then 72° C., 10 min). The 196-bp fragment was purified on a 2% agarose gel (Qiaquick gel extraction kit, Qiagen) and then cleaved, in a volume of 30 μl, with 5 U of PstI and 5 U of EagI in the presence of BSA, for 16 h at 37° C. The enzymes are destroyed for 15 min at 65° C. and the cleaved DNA fragment (168 bp) is extracted with phenol/chloroform and then precipitated with ethanol. The DNA fragment is taken up with 10 μl of ultrapure H₂O and then verified on a 2% agarose gel.

Preparation of the Vector:

Two μg of phagemid pHen1 are cleaved, in a volume of 30 μl, with 5 U of PstI and 5 U of EagI in the presence of BSA, for 16 h at 37° C. The cleaved phagemid is purified on a 0.7% agarose gel (Qiaquick gel extraction kit, Qiagen). The enzymes are destroyed for 15 min at 65° C. and the DNA is extracted with phenol/chloroform and then precipitated with ethanol. The cleaved phenyl is verified on a 0.7% agarose gel, quantified and taken up in 10 μl of ultrapure H₂O.

Ligation:

One μl of phenyl cleaved with PstI and EagI is ligated with 1 μl of fragment cleaved with PstI and EagI, in a volume of 10 μl, with 200 U of T4 DNA ligase (Biolabs) at 16° C. for 17 h. The ligase is inactivated at 65° C. for 15 min, and the ligation product is cleaved with 5 U of XhoI at 37° C. for 4 h in order to eliminate the residual nonligated vector, and then extracted with phenol/chloroform, precipitated in the presence of 1 μg of glycogen and taken up in 10 μl of ultrapure H₂O. One μl is used to transform E. Coli TG1 cells made competent by the CaCl₂ technique.

Verification of the clones expressing the SH3 domain:

The presence of the fragment inserted into the pHen1 plasmid is verified using the colonies isolated after transformation of the TG1 cells by carrying out DNA minipreparations. The recombinant plasmid pHen1SH3Hck is cleaved with PstI and EagI in order to verify the size of the inserted fragment. Some clones which have the inserted fragment are sequenced on an ABI 310 sequencer using the Fuse3p oligonucleotide of sequence SEQ ID No. 24 (5′CCCTCATACTTAGCGTAACG) which hybridizes in the region encoding the p3 protein. A clone having the correct sequence is selected in order to verify the production of the SH3-p3 fusion protein. For this, an isolated colony is inoculated into 3 ml of 2YT/100 μg/ml ampicillin/2% glucose and incubated at 30° C. with shaking. When the culture reaches an OD600 nm of 0.5, the cells are induced with a final concentration of 0.1 μm of IPTG (isopropyl-α-D-thiogalactopyranoside) and the culturing is continued at 30° C. for 16 h. An aliquot of the culture is taken and loaded onto an SDS/PAGE 10% gel. The presence of the SH3-p3 fusion protein is revealed by Western blotting with the monoclonal antibody 9E10 which recognizes the c-myc tag located between the SH3 domain and the p3 protein.

This control phage, called phage-SH3, is used as a positive control in ELISAs where the biotinylated GST-Nef_{Δ1-57} or Nef_{Δ1-57} protein is adsorbed in microplate wells.

2. Construction of a library of decameric peptides

A decameric library with a diversity of 10⁸ clones was constructed by insertion of degenerate oligonucleotides into a phage vector. To this end, the fd-tet-dog1 vector (Hoogenboom et al., 1991) was used, this vector exhibiting tetracycline resistance and comprising all the genetic support necessary for the synthesis of bacteriophages. Degenerate inserts encoding 10 amino acids were introduced upstream of the sequencing encoding the minor protein of the phage capsid, the p3 protein.

The cloning site is located between the signal sequence of the p3 protein and the p3 protein of the phage. The insert was chosen so as to conserve the nucleotide sequences encoding the amino acids located downstream of the signal sequence, in order to optimize the enzyme cleavage by the endogenous peptidase. The randomized part (NNK)₁₀ was chosen so as to limit the presence of STOP codons.

Preparation of the Cloning Vector:

The replicative form (RF) of the fd-tet-dog phage was purified on a cesium gradient according to the protocol described in Maniatis et al. (1982). Five hundred micrograms of RF were cleaved with 700 U of ApaI or NotI restriction enzymes (NE Biolabs, MA, USA) and purified by extraction with phenol and then precipitation with ethanol.

Preparation of the fragments to be inserted:

Sequences of the oligonucleotides SEQ ID Nos 25 and 26:

SEQ ID No. 25:
5′ CGTCATACCTTCGATCAACCACAGTGCACAG
SEQ ID No. 26:
5′ CTTCAACAGTTTCTGCCGCCGCACCACC(MNN)₁₀CTGTGCACTGTG
CTTGAT

Two hundred picomoles of each of the oligonucleotides of SEQ ID Nos 25 and 26 are hybridized in a final volume of 100 μl containing 1 mM of dNTP and 20 U of Dynazyme (Finnzymes, Helsinki, Finland), and denatured at 95° C. for 3 min. An extension is carried out by means of 30 successive cycles (48° C., 1 min and 72° C., 1 min). The extension product is treated twice with phenol/chloroform, precipitated with ethanol, and cleaved with 10 U of each of the ApaI and NotI enzymes for 16 h at 37° C. The DNA fragments are then purified by electrophoresis on a 15% polyacrylamide gel. To this end, the band corresponding to the expected molecular weight is excised and purified by diffusion in PBS for 16 h at 20° C. with stirring.

Ligation:

Five micrograms of fragments are subsequently ligated with 300 micrograms of vector in the presence of 6 U of ligase (NE BioLabs, MA, USA) in a final volume of 200 μl for 16 h at 20° C. The ligation product is extracted with phenol, precipitated with ethanol and taken up with 300 μl of TE. Forty μl of XL1-blue cells are electroporated with 2 μl of the ligation product, using a micropulser (Bio-Rad, CA, USA) at 1700 volts/cm, 200 ohms, 25 μF for 5 msec and 0.1 cm cuvettes. The cells are subsequently incubated for 1 h at 37° C. in 1 ml of 2YT containing 20 pg/ml of tetracycline, and then plated out on agar/2YT/tetracycline dishes and incubated for 16 h at 37° C. One hundred and fifty electroporations were thus carried out.

The diversity of the library was verified by sequencing the DNA of about a hundred clones using the Fuse-3p oligonucleotide primer SEQ TD No. 24 (CCCTCATAGTTAGCGTAACG) by means of an ABM Prism sequencer (Applied Biosystems, CA, USA). The library obtained is a library of approximately 10⁸ different clones.

3. Selection of Nef_{Δ1-57}-binding peptides by phage display

The Nef_{Δ1-57}-binding peptides were selected by the phage-display technique using the decameric peptide library constructed as indicated above.

Biotinylation of the Nef_{Δ1-57} protein

Five hundred μg of the Nef_{Δ1-57} protein are dialyzed against PBS for 16 h at 4° C. and biotinylated with biotin according to the recommendations of the manufacturer (Biotin Protein Labeling Kit, Roche Diagnostic, Basle, Switzerland). The concentration of the biotinylated protein is measured by colorimetry (Kit Biorad, CA, USA). The biotinylation efficiency is verified by ELISA using microplates adsorbed with streptavidin (ThermoLabsystem, Helsinki, Finland) and with the protein being revealed with an anti-Nef monoclonal antibody (MATG0020, transgene) and an anti-mouse antibody secondary antibody coupled to alkaline phosphatase.

Production of peptide-phages

An aliquot of the library (XL1-blue cells containing the peptide-phages) is incubated for 16 h at 37° C. in 500 ml of 2YT/20 pg/ml tetracycline with shaking, and then centrifuged twice at 6000 g for 10 min at 4° C. The culture supernatant containing the peptide-phages is precipitated with 1.5 vol of 16.7% (weight/volume) of PEG 8000/3.3 X NaCl for 16 h at 4° C., and then centrifuged at 12 000 g for 20 min at 4° C., and the pellet is taken up with 50 ml of PBS (0.14×NaCl; 0.01× phosphate buffer, pH 7.4). A second precipitation is carried out under the same conditions, but for 1 h. The pellet is taken up with 1 ml of PBS. The solution is filtered through a 0.45 lam filter and conserved at 4° C. It contains approximately 1013 peptide-phages.

Selection of peptide-phages

Three or four rounds of selection and amplification are carried out in order to isolated the Nef_{Δ1-57}-specific peptide-phages.

At each round, 20 μg of biotinylated Nef_{Δ1-57} are incubated with 10¹¹ phages (10 μl) in 500 μl of PBS containing 4% (weight/volume) of skimmed milk powder (PBS/milk) for 1 h at 20° C. with shaking. One mg of streptavidin-coated magnetic beads (Dynabeads M-280 Streptavidin; Dynal Biotech, Oslo, Norway) is preincubated for 1 h at 20° C. with PBS/milk and then added for 30 min at 20° C. with shaking. The beads are washed H times with PBS/milk, 5 times with PBS/0.1% Tween-20 and 5 times with PBS and finally resuspended with 100 μl of PBS. The peptide-phages are amplified by infecting TG1 bacterial cells (Δ(lac-pro), supE, thi, hsdD5/F′, traD36, proAB, lacI^{q}, lac ZΔM15) in 100 ml of 2YT/20 μg/ml tetracycline for 16 h at 37° C. A portion is plated out onto a agar/2YT/tetracycline dishes so as to obtain isolated colonies.

After 3 or 4 rounds of selection/amplification, the isolated colonies are cultured in microplate wells (Nunclon, Milian, Geneva, Switzerland) for 16 h at 37° C. The plates are then centrifuged (1000 g) and the supernatants containing the peptide-phages are analyzed by ELISA so as to determine their specificity, The DNA regions of the phages positive by ELISA, corresponding to the region encoding the peptides, were sequenced.

Five different sequences, SEQ ID Nos 3 to 7, were obtained and made it possible to define a consensus sequence SEQ ID No. 2 (FIG. 1).

4. Peptide synthesis

The five decameric peptides SEQ ID Nos 3 to 7 with the highest affinity for Nef, selected by the phage-display technique described above, were synthesized. Other peptides of varied sizes, SEQ ID Nos 8 to 21, analogous or homologous to the consensus motif discovered, were then also synthesized using the following general method.

Peptides of “H₂N-aaₙ- . . . aa₁-CONH₂” type were thus prepared in a semi-automatic solid-phase manner, using an ACT 400 automated parallel-chemistry synthesis apparatus having 40- and 96-well plates.

The solid support used is a Rink-Amide 100-200 mesh resin for automatic synthesis by a conventional fmoc strategy.

Completely automatically, the first fmoc-amino acid aa₁ is firstly attached to the solid support (100 to 200 mg of resin per well). For the coupling, the automated apparatus dispenses the following solutions into each well: (i) a 0.5 M solution of HBTU in DMF, (ii) a 1 M solution of N-methylmorpholine in DMF, and (iii) a solution of aa₁ at 0.5 M in NMP. The reaction mixture is agitated for 90 minutes and then a series of washes (DMF, MeOH, DCM, DMF) is carried out automatically before proceeding with a double coupling with the same amino acid. The side chain of the first amino acid and also that of all the amino acids that will be incorporated during the synthesis are continuously protected with various conventional acids labile protective groups, until final detachment of the peptide.

Secondly, and still automatically, the first grafted amino acid is deprotected in the N-terminal position of its fmoc function using a 20% solution of piperidine in dichloromethane. After various successive washes, the first amino acid is then coupled to the next amino acid, the amino-terminal part of which is protected with an fmoc group. For the coupling, the automated apparatus dispenses the following solutions into each well: (i) a 0.5 M solution of HBTU in DMF, (ii) a 1 M solution of N-methylmorpholine in DMF, and (iii) a solution of aa₂ at 0.5 M in NMP. The reaction mixture is stirred for 90 minutes, and then a further series of washes is carried out before proceeding with a double coupling with the same amino acid. The cycles of deprotecting the fmoc group and coupling the next amino acid are then repeated automatically until coupling and deprotection of the final amino acid aaₙ.

In a final step, cleavage is carried out semi-automatically with a 95/2.5/2.5 TFA/H₂O/TIS solution with stirring for 2 hours. The peptides are then precipitated with ether and centrifuged, and the supernatant is then eliminated. The process is repeated 3 times, and then the ether is evaporated off. The peptide is then dissolved in a 50/50 solution of H₂O/acetonitrile before being lyophilized.

This automated synthesis made it possible to design amide peptides. If desired, the acid analogs are prepared by using a resin of PS-2-chlorotrityle type. The purity of the peptides synthesized is evaluated by LC-MS coupling. If required, the peptides are purified by preparative HPLC.

In order to evaluate the cell penetration of the peptides and their subcellular localization, biotinylated peptides which may subsequently be detected using a suitable probe (for example, streptavidin-FITC) were synthesized. These peptides are first of all solid-phase synthesized semi-automatically by the fmoc strategy described above.

However, before cleavage of the resin, the N-terminal amino function is deprotected and conventional manual peptide coupling is carried out with biotin (Sigma-Aldrich®, ref. 86, 164-2). The cleavage of the resin corresponds to the final step, making it possible to detach the labeled peptide from the solid support. The purity of the biotinylated peptide is analyzed by HPLC, LC-MS. A purification step by preparative HPLC may subsequently be carried out depending on the purity observed.

5. Peptide specificity

ELISAs were developed, according to which:

either the Nef_{Δ1-57} protein fused to GST is directly adsorbed in the microplate wells,
or the Nef_{Δ1-57} protein cleaved from the GST protein with thrombin is biotinylated, and then adsorbed in microplate wells coated with streptavidin (FIG. 2). A first positive control consisted in incubating the well with an anti-Nef monoclonal antibody (MATG0020) and in revealing the Nef_{Δ1-57}/anti-Nef interaction with a peroxidase-labeled secondary antibody against mouse antibodies. A second positive control uses the SH3-phage. After addition to wells adsorbed with Nef_{Δ1-57}, the SH3-phage was incubated with an anti-phage (p8 protein of the phage) mAb, and then the interaction was revealed with an alkaline phosphatase-labeled secondary antibody against mouse antibodies.

For the competition ELISAs, the Nef_{Δ1-57} or GST-SH3 competitors or the synthetic peptides are added at various concentrations to the phages.

The peptide-phages 07B2S3 (▪) and 08B2s3 (♦) derived from the decameric library and the control phage SH3-Hck () bound to Nef_{Δ1-57} are characterized in the same way by means of a competition ELISA (FIG. 3). The wells adsorbed with biotinylated Nef_{Δ1-57} (FIG. 3A) or GST-Nef_{Δ1-57}) (FIG. 3B) are incubated with 10¹¹ single phages, and then with various amounts of Nef_{Δ1-57} or the SH3 domain of Hck fused to the GST protein, used as competitor. Very small amounts of Nef_{Δ1-57} are sufficient to displace the Nef_{Δ1-57}/peptide-phage interaction and reveal IC50 values of the order of one micromolar. Equivalent results are obtained with the other peptide-phages.

The affinity and the specificity of the peptide-phages were also determined by means of a competition ELISA using synthetic peptides (FIG. 4). The peptide-phage 08B2S3 (♦) derived from the decameric library and the control phage SH3-Hck () bound to Nef_{Δ1-57} are displaced in a competition ELISA with synthetic peptide 2. Equivalent results are obtained with the other peptide-phages and the other peptides. The other peptides described in FIG. 1 give a curve that is entirely equivalent to that obtained with peptide 2. IC50 values of the order of a micromolar were in fact measured.

The specificity of the peptides can also be determined directly from cell extracts. Lysates of COS-7 cells expressing Nef are incubated with the GST-SH3^{Hck} fusion protein in the presence of increasing amounts of the peptide. The mixture is then loaded onto a GST-specific affinity column. The column is then washed and the extract eluted is analyzed by SDS-PAGE and anti-Nef immunoblotting. An IC50 value is thus deduced for the peptides that inhibit the Nef-SH3 interaction in a native Nef protein cellular context.

6. Cellular activity of the peptides: mammalian-cell double hybrid and FACS

A cell assay based on the double-hybrid principle adapted to mammalian cells was developed, making it possible to evaluate the cellular activity of the peptides capable of penetrating the plasma membrane. This assay makes it possible to integrate the cellular toxicity and bioavailability parameters by means of quantitative and functional reading of the interaction between two protein partners.

The Checkmate™ commercial system (Promega) was adapted to the HIV-1 (Lai) Nef/SH3-Hck pair in COS-7 cell culture (FIG. 5). This system combines the expression of firefly luciferase under the control of the interaction between Nef and the SH3 domain of Hck, and the expression, which is independent, of renilla luciferase, a control for cell viability. The ratio of the intensity of these two luciferases makes it possible to quantify the activity of a Nef-SH3-interaction competitor capable of penetrating into the cell and the half-life of which is sufficient over the time period of the analysis. Other protein interactions are also reconstituted in this assay: one, which is non-SH3 (between Id and MyoD), in order to exclude the compounds having a nonspecific activity on the luciferase expression, and the other between SH3 of Hck and the SAM68 protein, in order to identify the compounds which interact via the SH3 domain and not via Nef.

The cells are transferred using Fugene6 (Roche), according to the distributor's protocol. Briefly, the plasmids PG5 (300 ng), pAct or pActNef (400 ng), pBindHckmutated or pBindHck (100 ng) and pbcks (200 ng) are mixed, and then the Fugene 6 (3 μl diluted in 100 μl of DMEM culture medium) is added. The plasmids/Fugene6 mixture is left to react for a period of 15 min at ambient temperature, and is then deposited dropwise into the cell culture well.

After expression for 24H, the cells are harvested by treatment with trypsin (Gibco, ref 25300-054), washed, and then dispensed into 96-well plates (reference 353072, Beckton Dickinson). 50 μl of DMEM culture medium are then added to each well.

The activity of a candidate inhibitor compound is tested by adding 25 μl of this diluted compound to the well already containing 50 μl of the cell culture, and the luciferase activity is determined 24 H later in each well using the DualGlo assay kit according to the recommendations of the supplier (Promega).

In this assay, the peptide of sequence ID No. 11 (W P A W L P) does not show any notable biological activity at low concentration compared with a control molecule, this being the case for concentrations ranging from 10 to 50 μM of the peptide (FIG. 5B), and under various experimental conditions alternating, in particular, the time at which and the period for which the peptide is added.

This peptide synthesized in a biotinylated form did not make it possible to visualize any cell penetration in an assay using secondary labeling by means of a probe coupled to a fluorochrome, followed by confocal microscopy analysis, suggesting that the peptide does not spontaneously penetrate the plasma membrane and/or is rapidly degraded or exported into the extracellular medium.

Further peptides were synthesized by incorporating, at the N-terminal position of sequence ID No. 11, various peptide sequences or sequences of amino acid derivatives (SEQ ID No. 14 to SEQ ID No. 19) rich in basic residues and known to allow the translocation of fusion sequences through the cell plasma membrane (FIG. 5B).

Several of these modified sequences (SEQ ID No. 15, SEQ ID No. 17 and SEQ ID No. 19) repress the Nef-SH3 interaction as measured in the cell assay, in a dose-dependent manner, showing that said sequences effectively penetrate through the cell plasma membrane (FIG. 5B).

7. Mapping the region of interaction of the peptides on Nef

¹⁵N-¹H HSQC spectra for the ¹⁵N-Nef protein were recorded on a 500 MHz NMR spectrometer (Bruker DRX 500) at 308 K. The conditions for sample preparation are identical to those used by Grzesiek et al. in 1997, for assignment of heteronuclear NMR spectra. The Nef concentration in the samples is 0.1 mM (FIG. 6), with 5 mM Tris, pH 8.0, 5 mM DTT.

The assignment of the correlations corresponding to the group of each of the NH of the protein is identical on the spectrum. Peptides 2, 4, 5 and 9c are taken up in 20 μl of deuterated ethanol at a concentration of 5 mM. The peptides are added in two-fold excess (10 μl) over the Nef (FIGS. 7A to 7D). The unlabeled peptide cannot be observed in the spectrum, only the ¹H/¹⁵N correlation peaks for the amide groups of each of the amino acids of the protein can be observed. The reference spectrum is the Nef spectrum in the presence of 10 μl of deuterated ethanol. The addition of peptide will result in a modification of the resonance frequency of the proton and/or ¹⁵N of the amide groups located in the region of interaction. Since the assignment of these resonances is known, the amino acids involved in the region of interaction can then be deduced. The nature of these spectral variations makes it possible to verify whether the region of interaction indeed corresponds to that of the SH3 domain of Hck. The analysis of these data confirms that the peptides tested indeed interact on Nef in the SH3 interaction region; furthermore, the differences observed between the spectra in the presence of the various peptides indicate slightly different binding geometries.

8. Structural studies by crystallography

The “dip” method is used to determine the structure of the peptides in a complex with Nef. For this, crystals of Nef_{Δ1-56} and Nef_{Δ1-57} were produced as previously described (Arold et al., 1997). After having reached a sufficient size for crystallographic analysis (>100 lam), these crystals are dipped for 1-24 h in a solution containing between 1 and 5 mM of peptide. The presence of solvent channels and also the arrangement of the Nef_{Δ1-56} and Nef_{Δ1-57} molecules in this crystalline form in fact provide small molecules with access to the Nef-peptide region of interaction mapped by NMR (described above). The crystals thus prepared are analyzed by X-ray diffraction.

9. Virology

The study of the positive influence of Nef on viral replication is carried out by conventional techniques for measuring the infectious capacities of HIV-1 (Craig et al., 1998; Madrid et al., 2005). The initial analyses are carried out using a prototype laboratory provirus, HIV-1 pNL4-3. Briefly, the effect of the Nef-inhibiting peptides will be evaluated on the virions produced by transient transfection of 293T cells with the wild-type provirus and used to infect HeLa-CD4 target cells (clone P4) containing one integrated copy of the LacZ gene under the control of the HIV-1 LTR. 48 h after infection, the infectious capacity of the viruses is evaluated by counting the cells expressing β-galactosidase activity.

### LITERATURE REFERENCES

Arold S., Franken P, Strub M. P., Hoh F., Benichou, S. Benarous, R., and Dumas, C. (1997). The crystal structure of HIV-1 Nef protein bound to the Fyn Kinase SH3 domain suggests a role for this complex in altered T cell receptor signalling. Structure 5, 1361-72.
Craig, H. M., Reddy, T. R., Riggs, N. L., Dao, P. P., and Guatelli, J. C. (2000). Interactions of HIV-1 nef with the mu subunits of adaptor protein complexes 1, 2, and 3: role of the dileucine-based sorting motif. Virology 271, 9-17.
Grzesiek, S. Bax, A., Hu, J, Kaufman, J., Palmer, I., Stahl, S., Tjandra, N. and Wingfield, P. T. (1997) Refined solution structure and backbone dynamics of HIV-1 Nef. Protein science, 6, 1248-1263.
Hoogenboom H R, Griffiths A D, Johnson Kans., Chiswell D J, Hudson P, Winter G. Multi-subunit proteins on the surface of filamentous phage: methodologies for displaying antibody (Fab) heavy and light chains Nucleic Acids Res, 1991, 19: 4133-4137.
Madrid, R., Janvier, K., Hitchin, D., Day, J., Coleman, S., Noviello, C., Bouchet, J., Benmerah, A., Guatelli, J. and Benichou, S. (2005) Nef-induced alteration of the early/recycling endosomal compartment correlates with enhancement of HIV-1 infectivity. J. Biol. Chem., 280: 5032-5044.
Maniastis T., Fritsch E. F., and Sambrook J. (1982), Molecular Cloning: A Laboratory Manual, Cold Spring Harbor Laboratory Press, Cold Spring Harbor, N.Y.
McLoughlin, P, Ehler, E., Carlile, G., Licht, J. D., and B. W. Schafer. The LIM-only Protein DRAL/FHL2 Interacts with and Is a Corepressor for the Promyelocytic Leukemia Zinc Finger Protein. J. Biol. Chem. 2002, 277: 37045-53.

1. A purified isolated peptide, having in particular Nef-protein-binding properties, characterized in that it contains an amino acid sequence corresponding to SEQ ID No. 1:
W-P-a-W-L-P
wherein
a is selected from W, A, S or D.

2. The peptide as claimed in claim 1, characterized in that it corresponds to the amino acid sequence SEQ ID No. 2:
b-W-P-a-W-L-P-c-d-f
wherein
b=R/T or is absent
a is as defined above
c=Q, T, L, G or H or is absent
d=L, W or A or is absent
f=P or is absent.

3. The peptide as claimed in claim 1, characterized in that it is a decameric peptide corresponding to the following sequences SEQ ID No. 3 to SEQ ID No. 7:

| | |
|---|---|
| SEQ ID No. 3: | N T W P W W L P T L |
| SEQ ID No. 4: | Y R W P A W L P L W |
| SEQ ID No. 5: | N W R W P W W I P G |
| SEQ ID No. 6: | T W P W W L P H A P |
| SEQ ID No. 7: | W P S W L P Q L P F |

4. The peptide as claimed in claim 1, characterized in that it corresponds to the following sequences SEQ ID No. 8 to SEQ ID No. 13:

| | |
|---|---|
| SEQ ID No. 8: | W P S W L P Q |
| SEQ ID No. 9: | W P S W L P |
| SEQ ID No. 10: | W P W W L P |
| SEQ ID No. 11: | W P A W L P |
| SEQ ID No. 12: | W P D W L P |
| SEQ ID No. 13: | W P S W L P Q L P. |

5. The peptide as claimed in claim 1, characterized in that it contains an amino acid sequence comprising, where appropriate, amino acid derivatives which facilitate the penetration of said peptide into cells, said sequence being selected from:
(R)ₙ with n=6 to 8; R (A R R)ₙ₁, with n1=1 to 3; R (Ahx R)ₙ₂, with n2=1 to 6;
SEQ ID No. 14: K K R R Q R R R; and SEQ ID No. 15: R Q I K I W F Q N R Nle K W K K, “Ahx” representing an aminohexanoic acid unit and “NIe” representing norleucine.

6. The peptide as claimed in claim 5, characterized in that it is a derivative of sequence ID No. 11, and in that it corresponds to the following sequences SEQ ID No. 16 to SEQ ID No. 21:

| | |
|---|---|
| SEQ ID No. 16: | R R R R R R W P A W L P |
| SEQ ID No. 17: | R R R R R R R R W P A W L P |
| SEQ ID No. 18: | R A R R A R R A R R W P A W L P |
| SEQ ID No. 19: | R Ahx R Ahx R Ahx R Ahx R Ahx R |
| | Ahx R W P A W L P |
| SEQ ID No. 20: | K K R R Q R R R W P A W L P |
| SEQ ID No. 21: | R Q I K I W F Q N R Nle K W K K W |
| | P A W L P. |

7. The use of the peptide as claimed in claim 1, as inhibitors of the interaction between Nef and some of its cellular partners, including SH3-domain proteins.

8. The use of the peptide as claimed in claim 1, for developing chemical molecules on the basis of its amino acid sequences and/or of its structural data.

9. A pharmaceutical composition characterized in that it comprises a therapeutically effective amount of at least one peptide as defined above, combined with pharmacologically acceptable excipients.


SEQUENCE LISTING (27 sequences)

SEQ ID NO: 1 - protein, 6 aa, Artificial Sequence
  Description of Artificial Sequence Synthetic peptide
WPXWLP

SEQ ID NO: 2 - protein, 10 aa, Artificial Sequence
  Description of Artificial Sequence Synthetic peptide
XWPXWLPXXX

SEQ ID NO: 3 - protein, 10 aa, Artificial Sequence
  Description of Artificial Sequence Synthetic peptide
NTWPWWLPTL

SEQ ID NO: 4 - protein, 10 aa, Artificial Sequence
  Description of Artificial Sequence Synthetic peptide
YRWPAWLPLW

SEQ ID NO: 5 - protein, 10 aa, Artificial Sequence
  Description of Artificial Sequence Synthetic peptide
NWRWPWWIPG

SEQ ID NO: 6 - protein, 10 aa, Artificial Sequence
  Description of Artificial Sequence Synthetic peptide
TWPWWLPHAP

SEQ ID NO: 7 - protein, 10 aa, Artificial Sequence
  Description of Artificial Sequence Synthetic peptide
WPSWLPQLPF

SEQ ID NO: 8 - protein, 7 aa, Artificial Sequence
  Description of Artificial Sequence Synthetic peptide
WPSWLPQ

SEQ ID NO: 9 - protein, 6 aa, Artificial Sequence
  Description of Artificial Sequence Synthetic peptide
WPSWLP

SEQ ID NO: 10 - protein, 6 aa, Artificial Sequence
  Description of Artificial Sequence Synthetic peptide
WPWWLP

SEQ ID NO: 11 - protein, 6 aa, Artificial Sequence
  Description of Artificial Sequence Synthetic peptide
WPAWLP

SEQ ID NO: 12 - protein, 6 aa, Artificial Sequence
  Description of Artificial Sequence Synthetic peptide
WPDWLP

SEQ ID NO: 13 - protein, 9 aa, Artificial Sequence
  Description of Artificial Sequence Synthetic peptide
WPSWLPQLP

SEQ ID NO: 14 - protein, 8 aa, Artificial Sequence
  Description of Artificial Sequence Synthetic peptide
KKRRQRRR

SEQ ID NO: 15 - protein, 15 aa, Artificial Sequence
  Description of Artificial Sequence Synthetic peptide
RQIKIWFQNRXKWKK

SEQ ID NO: 16 - protein, 12 aa, Artificial Sequence
  Description of Artificial Sequence Synthetic peptide
RRRRRRWPAWLP

SEQ ID NO: 17 - protein, 14 aa, Artificial Sequence
  Description of Artificial Sequence Synthetic peptide
RRRRRRRRWPAWLP

SEQ ID NO: 18 - protein, 16 aa, Artificial Sequence
  Description of Artificial Sequence Synthetic peptide
RARRARRARRWPAWLP

SEQ ID NO: 19 - protein, 19 aa, Artificial Sequence
  Description of Artificial Sequence Synthetic peptide
RXRXRXRXRXRXRWPAWLP

SEQ ID NO: 20 - protein, 14 aa, Artificial Sequence
  Description of Artificial Sequence Synthetic peptide
KKRRQRRRWPAWLP

SEQ ID NO: 21 - protein, 21 aa, Artificial Sequence
  Description of Artificial Sequence Synthetic peptide
RQIKIWFQNRXKWKKWPAWLP

SEQ ID NO: 22 - DNA, 31 bp, Artificial Sequence
  Description of Artificial Sequence Synthetic oligonucleotide
aatgcaaaactgcaggtggttgccctgtatg

SEQ ID NO: 23 - DNA, 31 bp, Artificial Sequence
  Description of Artificial Sequence Synthetic oligonucleotide
tttgttctgcggccgcgtcaacgcgggcgac

SEQ ID NO: 24 - DNA, 20 bp, Artificial Sequence
  Description of Artificial Sequence Synthetic oligonucleotide
ccctcatagttagcgtaacg

SEQ ID NO: 25 - DNA, 31 bp, Artificial Sequence
  Description of Artificial Sequence Synthetic oligonucleotide
cgtcataccttcgatcaagcacagtgcacag

SEQ ID NO: 26 - DNA, 76 bp, Artificial Sequence
  Description of Artificial Sequence Synthetic oligonucleotide
cttcaacagtttctgcggccgcaccaccmnnmnnmnnmnnmnnmnnmnnmnnmnnmnnctgtgcactgtgcttgat

SEQ ID NO: 27 - protein, 10 aa, Artificial Sequence
  Description of Artificial Sequence Synthetic peptide
XWPWWLPXLP